(19) United States
(12) Patent Application Publication
(10) Pub. No.: US 2014/0277313 A1
(43) Pub. Date: Sep. 18, 2014

(54) Implantable Electrode

(71) Applicant: BIOTRONIK SE & Co. KG, Berlin (DE)

(72) Inventor: Gernot Kolberg, Berlin (DE)

(73) Assignee: BIOTRONIK SE & Co. KG, Berlin (DE)

(21) Appl. No.: 14/171,304

(22) Filed: Feb. 3, 2014

Related U.S. Application Data
(60) Provisional application No. 61/781,110, filed on Mar. 14, 2013.

(51) Int. Cl. A61N 1/05

(52) U.S. Cl. CPC A61N 1/05
USPC 607/116

(57) ABSTRACT

An implantable electrode includes an elongate electrode body and at least one lumen which extends therein at least over a portion of the length of the electrode body. A pressure device reversibly supplies a pressure medium to the at least one lumen such that the electrode body varies the flexibility and/or shape thereof depending on the pressure of the pressure medium.

### CROSS-REFERENCE TO RELATED APPLICATIONS

This patent application claims the benefit of co-pending U.S. Provisional Patent Application No. 61/781,110, filed on Mar. 14, 2013, which is hereby incorporated by reference in its entirety.

### TECHNICAL FIELD

The present invention relates generally to implantable electrodes and, in particular, an implantable electrode comprising an elongate electrode body and at least one lumen which extends therein over at least a portion of the length of the electrode body.

### BACKGROUND

In general, such electrodes are intended for implantation in or on the heart, in vessels, in or on muscles, in the brain, spinal cord, along nerve cords, and the like. Depending on the application purpose, they can be provided with electrode surfaces for electrophysiological stimulation and/or for the corresponding sensing of electrophysiological body signals and/or, provided with sensors for picking up any physiologically relevant information, and/or provided with actuators for manipulation in the body or for delivering medication.

The problem(s) addressed by the present invention shall be described hereafter based on the example of a cardiac electrode. So as to allow the electrode to achieve an optimal effect according to the therapeutic purpose, exact placement of the electrode in the heart is important. In particular during transluminal insertion, the electrode must exhibit a certain degree of axial stability. In addition, it may be helpful for certain target sites in the body if the electrode is able to variably form an arc or exhibits the same already in a predefined shape.

The rigidity associated with these mechanical properties is problematic, notably with a permanently implanted electrode. For the long-term application, such electrodes should be as soft as possible so as to pose as little mechanical stress on the heart as possible. A flexible electrode generally also has increased long-term stability.

Various techniques are known from the prior art for solving the problem(s) described above. For example, the electrode may be stiffened during implantation by means of a wire—referred to as a stylet or mandrin. For this purpose, the electrode has an inner lumen, which can receive the wire for the implantation procedure. This stiffens the electrode, allowing it to be inserted into the vessels. The wire can additionally be pre-bent, whereby it imparts a shape(s) to the electrode if needed. Because a wire can be pulled quickly out of the electrode body, deformation can be varied very easily and adapted to the corresponding needs. In addition, embodiments are known from the prior art in which the bending of the wire can be varied during the application.

Another known approach lies in the use of pre-shaped electrodes, for example, so-called J-electrodes. These are particularly easy to anchor in the atrium of the heart because they are provided, in the distal region, with an imparted curve which bends the electrode into the desired position after a stiffening wire has been pulled.

It is further known to guide the electrode through a catheter. The sheath introducers in this case are conventionally short and only used to insert the electrode in the vascular system and guide it into a vessel at the inlet. Especially with applications in the left part of the heart, catheters having special curve shapes are used, which can be easily guided to the desired locations. The electrode can then be inserted through these catheters. In this connection, very thin electrodes are known, which dispense with a guide wire lumen. A special catheter is then required for the implantation.

An electrode can also be guided on a wire. For this purpose, a soft wire is used, which is pushed to a desired position with the aid of a catheter serving as a placeholder. An open electrode is then inserted to the corresponding site in the body via this wire.

For these auxiliary components, the aforementioned electrode types, which use a stylet, a mandrin or a guide wire, have a radially stabilized lumen which allows the electrode to slide smoothly over these components. The lumen is generally implemented by a helix, which additionally assumes the function of a supply line and, in the case of actively fixable electrodes, that of torsion transmission. However, this lumen cannot be eliminated after the implantation because the helix is rigidly integrated in the electrode body. After implantation, the electrode can thus not be designed more flexibly.

The rigidity of guide wires is also dependent on the diameters thereof. Due to a particular minimum diameter of such wires, it is also not possible to decrease the guide lumen provided therefor. The guide lumen thus proves to be the limiting variable for the desired diameter reduction of electrodes.

Electrodes which, due to the design thereof, have no guide lumen, require a complex guide catheter which is cumbersome to operate.

The present invention is directed toward overcoming one or more of the above-identified problems.

Proceeding from the problems of the prior art described above, it is an object of the present invention to design an implantable electrode that has a smaller diameter and is more flexible in long-term use, while offering good controllability that is adapted to the purpose of use. The electrode in particular is to be temporarily stabilized for the time of implantation, and optionally pre-shaped, without having to provide a permanently open lumen for a guide wire or requiring a catheter.

### SUMMARY

At least the above object is achieved in terms of the concept by the features provided in claim 1, according to which a pressure device is provided, by means of which a pressure medium can be reversibly supplied to the at least one lumen such that the electrode body varies the flexibility and/or shape thereof depending on the pressure of the pressure medium.

Based on this solution according to the present invention, advantageously thinner electrodes can be designed, because the cross-section of the lumen to which the pressure medium is to be supplied can be considerably smaller than a lumen for a guide wire. Because of the appropriate design of the lumen and the material regions of the electrode body surrounding the same, the functional spectrum of such electrodes—as will be shown in the advantageous refinements described in the dependent claims—can be considerably expanded, since the properties of the electrode can be varied for the time of the implantation procedure with considerably more flexibility than in the prior art. Moreover, new implantation aids can be incorporated into the products, which can considerably simplify the implantation procedure.

It detail, it is preferred for the at least one lumen to be filled with a liquid pressure medium, preferably with a sodium chloride solution or a suitable gel. The pressure system is thus hydraulic.

In a preferred embodiment, the lumen can form a closed hydraulic system together with a corresponding compressible, preferably manually actuatable, reservoir for the pressure medium. This reservoir can, for example, be located in the region of the proximal connector of the electrode and increase the pressure in the overall system by compression, for example, by pressure using the fingers or permanently by applying a ligature. During the implantation process, the electrode can thus be stiffened by the surgeon at any time without the use of tools by increasing the pressure, or it can be relaxed by lowering the pressure.

According to a further alternative embodiment, an injection device for the pressure medium can be detachably connected to the at least one lumen of the electrode. In practice, the mandrin access of a conventional connector for the pressure connection can be used for this purpose. Salt solution, for example, is then injected via the connected syringe using what is known as a Luer lock fitting and subjected to a defined pressure. Of course, other injection procedures and fittings are contemplated.

Varying the pressure by, for example, means of the afore-described measures can be utilized to change a variety of properties of the electrode body. For example, because of the pressure of the pressure medium, the at least one lumen can lead, in defined reinforcement regions, to an electrode body being stiffened in these regions as a function of the pressure. For safety reasons, the material surrounding the lumen can have a yield point in these stiffening regions, which preferably can be adjusted by a fiber or filler reinforcement of the material.

According to an alternative or additional preferred refinement, because of the pressure of the pressure medium, the at least one lumen, in defined expansion regions, leads to an electrode body having a changed shape in terms of the diameter, notably an increased or curved electrode body, in these regions as a function the pressure. This change in diameter or shape can preferably be implemented by a pleated lumen in the defined expansion regions, wherein the pleats can be deployed by the pressure of the pressure medium. The present invention is not, however, limited to this implementation mechanism.

All shape and flexibility changes have in common that they can be reversed by releasing the pressure, so that the electrode has a minimal diameter and maximum flexibility after the implantation procedure has been carried out.

Preferred materials for lining or enveloping the lumen are thin-walled, tear-resistant materials having a yield point, such as PET (polyethylene terephthalate) or PE (polyethylene), for example, which offer particularly good protection against tearing of the lumen wall. This allows good stiffening of the electrode body, wherein the lumen increases the diameter of the electrode body when it is pressurized, provided the final diameter has not yet been reached. If the lumen is provided with a thin-walled, tear-resistant material lining, but already has the final diameter without any pressurization, the lumen will stiffen under pressure, without increasing the diameter of the electrode body.

The aforementioned yield point of the material can preferably be adjusted in the stiffening regions of the lumen by, for example, a fiber or filler reinforcement of the material surrounding the lumen.

According to an additional or alternative refinement of the electrode body, because of the pressure of the pressure medium, the at least one lumen, in defined expansion regions, leads to an electrode body having a changed shape in terms of the diameter, notably an increased or curved electrode body, in these regions as a function of the pressure. It is advantageous for this purpose for the lumen to be in the expandable material of the electrode body in these expansion regions. Such material can, for example, include silicones, polyurethanes, copolymers, and the like. The deformation effect can then be controlled by varying the pressure.

Preferred configurations for the lumen design provide for the formation of a star-shaped cross-section of the lumen by the pleats, wherein the cross-section can transition into a circular expanded cross-section by supplying the pressure medium.

A further alternative for a deformation of the electrode body is that of providing the electrode body over a target curvature length with at least one eccentrically arranged lumen having a sickle-shaped cross-section. When such a lumen is subjected to pressure, it curves in the direction that is opposite the eccentricity of the lumen, so that an electrode is achieved which can be controlled or placed in curvature radii by the pressurization.

In general, a wide range of shape and property changes along the electrode body can be achieved by the way the lumens are arranged and the number thereof. For example, two or more different lumens or lumen sections, which are preferably connected to each other by pressure valves, can be provided in the electrode body. These individual lumens or lumen sections can then be selectively actuated via different pressure levels of the pressure medium, in particular, by means of the pressure valves, in that the pressure valves, for example, open at different pressure values. When the pressure increases and a lumen section thus actuated opens, the shape of the electrode can thus be deliberately changed.

If two or more lumens are located in material regions of the electrode body which exhibit differing expansion levels, the electrode can take on new shapes every time the pressure is increased.

According to a further preferred embodiment of the present invention, variable functional parts, such as, for example, hooks, screws or spirals for anchoring the electrode body in the surrounding body tissue can be formed in portions of the electrode body by supplying the pressure medium to the at least one lumen. The change of the electrode body's shape is thus helpful when positioning and fixing the electrode. For example, so-called tines can be temporarily stiffened so as to insert the electrode deeper into, for example, the trabeculae of the heart tissue. In another variant, small hooks can be folded forward and back during pressure fluctuations, which allow a so-called CS electrode to migrate forward in the vessel. A fixation of a screw triggered by pressurization is also conceivable.

Conversely, in a further preferred embodiment, the electrode body can be pre-shaped at least in some sections; for example, it can have a helical shape, which can be temporarily eliminated by applying pressure to the at least one lumen in the electrode body. For the implantation, for example, in the vascular system, it is possible to achieve temporary straightening of the electrode body due to the pressurization; the straightening being eliminated again after the pressure is released, whereby the pre-shaped configuration returns.

In general, it should be pointed out that an electrode according to the present invention may comprise electronic components for actuating and/or reading electrodes, sensors or actuators and/or for preprocessing or transmitting information or energy. These measures do not have anything to do with the core of the present invention per se and are therefore not reflected separately in the dependent claims. They are, however, contemplated by the present invention.

Further features, aspects, objects, advantages, and possible applications of the present invention will become apparent from a study of the exemplary embodiments and examples described below, in combination with the figures, and the appended claims.

### DESCRIPTION OF THE DRAWINGS

Further characteristics, details and advantages of the present invention will be apparent from the following description of exemplary embodiments based on the accompanying drawings. In the drawings:

FIG. 1 is an assembly drawing of a bradycardia electrode comprising a standard IS-1 connector, a pressure adapter therefor, and a pressure transducer;

FIG. 2 is an enlarged sectional view of the electrode body at position II according to FIG. 1 in the depressurized state;

FIG. 3 is a representation analog to FIG. 2 with pressurization;

FIG. 4 is an assembly drawing of an ICD electrode comprising a standard DF-4 connector and a pressure adapter therefor;

FIG. 5 is an enlarged sectional view of the electrode body at position V according to FIG. 4 in the depressurized state;

FIG. 6 is a representation analog to FIG. 5 with pressurization;

FIG. 7 is a view of a J-electrode comprising an IS-1 connector in the depressurized state;

FIGS. 8 and 9 are enlarged sectional views of the J-electrode at positions VIII and IX according to FIG. 7;

FIG. 10 is a representation of the J-electrode analog to FIG. 7 with pressurization to a first pressure level;

FIGS. 11 and 12 are enlarged sectional views of the J-electrode at positions XI and XII according to FIG. 10;

FIG. 13 is a view of the J-electrode according to FIGS. 7 and 10 with pressurization to a second pressure level, which is higher than the first pressure level according to FIG. 7; and

FIG. 14 is an enlarged sectional view of the J-electrode at position XIV according to FIG. 13.

### DETAILED DESCRIPTION

The bradycardia electrode 1 shown in FIG. 1 has an elongate, very thin electrode body 2 made of a suitable expandable, flexible material, such as a polymer like Polyurethane, Silicone Polyamide like Nylon, Polyethylene or Polyethylenterephthalat, for example, which—as is shown in FIGS. 2 and 3—guides two supply lines 3.1, 3.2 for the annular electrode 4 and tip electrode 5 at the distal end 6 of the electrode body 2 via lumens in the electrode body 2, which are not shown separately. The two supply lines 3.1, 3.2 are disposed eccentrically outside the longitudinal axis of the electrode body 2. Coaxially thereto, a lumen 7, which is provided with a thin, tear-resistant wall 8, is introduced in the electrode body 2.

As is further apparent from FIG. 1, the bradycardia electrode 1 is provided with a standardized IS-1 connector 9 for connecting the supply lines 3.1, 3.2, with the connector centrally having an outlet 10 for the lumen 7.

A pressure adapter 11 is associated as a kit with the electrode 1 and can be mounted on the outlet 10 of the IS-1 connector 9 in a pressure-tight manner. A Luer lock fitting 13, which can be connected to a PTCA pressure transducer 15 using a mating Luer lock piece, is connected by a hose 12 to the pressure adapter 11. A reservoir 16 for a pressure medium, such as, for example, a salt solution or a suitable gel, which can be pressurized by a spindle 17, is provided in this pressure transducer. The pressure in the reservoir 16 can be read by a pressure gauge 18.

As is apparent from FIG. 2, in the depressurized state, the lumen 7 is folded in plates F in a star-like shape, so that the electrode body 2 takes on a minimal diameter over the entire length and is very flexible. The electrode body 2 is thus in an optimal state for a permanent implantation in the body.

For the implantation procedure, it may be advantageous to stiffen the electrode 1 so as to achieve better insertion through the body vessels. So as to achieve this, the lumen 7 is filled with the pressure medium via the pressure transducer 15 and thus pressurized. The lumen 7 deploys, and the electrode body 2 expands, in the shown expansion region A (see FIG. 1) for the electrode body 2 and is considerably stiffened, which is shown in FIG. 3. The electrode 1 is thus temporarily stiffened for the implantation process. After completion thereof, the pressure medium can be suctioned out of the lumen 7 again, and the lumen 7 collapses, making the electrode body 2 again very thin and flexible.

FIGS. 4 to 6 show an ICD electrode as an exemplary embodiment of the present invention, in which the electrode body 2, instead of having only two lumens with respective supply lines 3.1, 3.2, has two additional lumens with supply lines 3.3, 3.4, which can be connected via a corresponding DF-4 connector 19. Two annular electrode surfaces 4.1, 4.2, and again a tip electrode 5, for example, can be electrically connected via these supply lines to a screw-in extension 20. Similarly to the exemplary embodiment of FIGS. 1 to 3, the central lumen 7 can be pressurized by a pressure adapter 11 and a balloon-shaped closed reservoir 15′. By means of pressurization via the balloon reservoir 15′, the lumen 7—as is shown in FIGS. 5 and 6—is to be transferred from the folded state (FIG. 5—pleats F) into a stiffened configuration in which the lumen 7 expands (FIG. 6).

FIGS. 7 to 14 show a further embodiment of the present invention based on a so-called J-electrode, which, similar to the embodiment according to FIGS. 1 to 3, has two supply lines 3.1, 3.2 to an annular electrode 4 and a tip electrode 5. This J-electrode 22 is again connected via an IS-1 connector 9, which leads the lumen 7 out via the central outlet 10 thereof.

As is apparent from FIG. 8, the electrode body 2 has a single lumen 7 at a position far away from the distal end 6 in accordance with the intersecting line VIII of FIG. 7, with the lumen being guided through to the distal end 6, as is apparent from FIG. 9.

The J-electrode 22 is characterized in that the electrode body thereof can be deformed over a length of several centimeters in front of the distal end 6 into a curved arc shape—thus a J-shape—as is indicated in FIG. 13. So as to be able to generate this arc shape in a reversible manner, a second lumen 23 branches off of the central lumen 7 in front of the arc to be generated, the second lumen having a sickle-shaped cross-section and running eccentrically with the sickle-shaped bend approximately parallel to the outside contour of the electrode body 2 having a circular cross-section, as is apparent from FIGS. 9 and 12. This second lumen 23 forms an expansion region A for the electrode body 2 and then ends at the distal end 6 in front of the annular electrode 4.

The pressure response behavior of the two lumens 7 and 23 is designed so that, starting from the depressurized state, first the central lumen 7 expands when a first pressure value P1 is applied and, thus, the J-electrode 22 stiffens over the entire length thereof. This state is shown in the sectional views according to FIGS. 11 and 12.

When the pressure is increased further to a value P2>P1, the pressure medium is introduced into the lumen 23 that branches off, so that the same is pumped from the non-expanded state shown in FIGS. 9 and 12 into the expanded state shown in FIG. 14. The associated volume expansion of the material of the electrode body 2 located on the side of the lumen 23 results in an overall expansion, so that the electrode body 2 bends into the J-shape shown in FIG. 13. This behavior is comparable to the curvature mechanism of a bimetallic strip. Because of the axially one-sided expansion of the electrode body 2, the body undergoes shaping in, for example, the 180° radius, which allows the electrode to be easily positioned in the atrium of the heart and fixed in place there.

After the pressure medium (for example, a salt solution) has been removed by suctioning, the electrode is again very thin and highly flexible.

The different response parameters of the two lumens 7 and 23 can, for example, be adjusted by wall materials having differing rigidities or by a pressure valve at the branching point of the lumen 23 from the lumen 7, the pressure valve not being shown in the drawings.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/We claim:

1. An implantable electrode, comprising:
an elongate electrode body;
at least one lumen, which extends therein over at least a portion of the length of the electrode body; and
a pressure device, by means of which the at least one lumen can be reversibly supplied with a pressure medium such that the electrode body varies the flexibility and/or shape thereof depending on the pressure of the pressure medium.

2. The electrode according to claim 1, wherein the at least one lumen is filled with a liquid pressure medium comprising a sodium chloride solution or a gel.

3. The electrode according to claim 2, wherein the at least one lumen filled with the liquid pressure medium forms a closed hydraulic system together with a compressible, manually actuatable reservoir.

4. The electrode according to claim 1, wherein an injection device for the pressure medium is configured for detachable connection to the at least one lumen of the electrode.

5. The electrode according to claim 1, wherein the at least one lumen, in defined stiffening regions, leads to an electrode body being stiffened in these defined stiffening regions as a function of the pressure of the pressure medium.

6. The electrode according to claim 5, wherein the material surrounding the lumen has a yield point in the stiffening regions, the yield point being adjustable by a fiber or filler reinforcement of the material.

7. The electrode according to claim 1, wherein the at least one lumen, in defined expansion regions, leads to an electrode body having a changed shape in terms of the diameter, the changed shape comprising an increased or curved electrode body, in these defined expansion regions as a function of the pressure of the pressure medium.

8. The electrode according to claim 7, wherein in the defined expansion regions, the at least one lumen is placed in pleats which are configured to be deployed by the pressure of the pressure medium.

9. The electrode according to claim 8, wherein the pleats form a substantially star-shaped cross-section of the lumen, which transitions into an expanded cross-section because of the pressure medium that is supplied thereto.

10. The electrode according to claim 1, wherein the at least one lumen is lined with a thin-walled, tear-resistant material.

11. The electrode according to claim 1, wherein the electrode body is provided over a target curvature length with at least one eccentrically arranged lumen having a sickle-shaped cross-section.

12. The electrode according to claim 1, wherein the electrode body comprises two or more different lumens or lumen sections, which are connected to each other by pressure valves and which are configured for selective actuation via different pressure levels of the pressure medium.

13. The electrode according to claim 1, wherein the electrode body comprises two or more lumens which are located in material regions of the electrode body which exhibit differing expansion levels.

14. The electrode according to claim 1, wherein functional parts for anchoring the electrode body in the surrounding body tissue form in portions of the electrode body by supplying the pressure medium to the lumen, wherein the functional parts comprise hooks, screws or spirals.

15. The electrode according to claim 1, wherein the electrode body is pre-shaped at least in some sections which can be temporarily eliminated by applying pressure to the at least one lumen, wherein the pre-shape comprises a helical shape.
